(19) United States
(12) Patent Application Publication
(10) Pub. No.: US 2008/0247972 A1
(43) Pub. Date: Oct. 9, 2008

(54) Mouthwash for the Prevention and Treatment of Halitosis

(76) Inventor: Mauricio Duarte da Conceicao, Sao Paulo (BR)

Correspondence Address: AMIN, TUROCY & CALVIN, LLP, 1900 EAST 9TH STREET, NATIONAL CITY CENTER, 24TH FLOOR,, CLEVELAND, OH, 44114, US

(21) Appl. No.: 11/916,274

(22) PCT Filed: Jun. 2, 2006
(86) PCT No.: PCT/BR2006/000109

§ 371 Date: Nov. 30, 2007

(30) Foreign Application Priority Data
Jun. 3, 2005 (BR) PI 0502145-6

(51) Int. Cl. A61K 8/22; A61K 8/30; A61Q 11/00

(52) U.S. Cl. 424/52; 424/53

(57) ABSTRACT

The present aspects relate to a mouthwash developed to reduce and/or avoid the formation of tonsilloliths, heretofore unknown to the art, to facilitate the removal and to diminish the new formation of tongue coating and to reduce the concentration and formation of volatile sulfur compounds. In a preferred aspect, the compound includes Hydrogen peroxide, Citric acid, flavorizer, Sodium benzoate, Cetylpyridinium chloride, colorant, Sodium fluoride, Methyl paraben, Polisorbate 20 (Tween 20), Dimethicone, Sodium saccharine, Sorbitol and demineralized water. The compound may be used in mouth washing and gargling so that the active ingredients can be released.

### FIELD OF THE INVENTION

The present description relates to an invention in the field of mouthwashes, particularly the ones that help treat and control of bad breath (whose medical term is halitosis). Through the present effects, it is possible to greatly reduce or avoid the formation of tonsilloliths, which heretofore was unknown to the art, to facilitate the removal and to diminish new formations of tongue coating and to reduce the concentration and formation of volatile sulfur compounds.

### BACKGROUND

Bad breath affects more than 30% of the Brazilian population and research data in other countries shows similar indexes worldwide.

Two of the most frequent causes of bad breath are found in the oral cavity and the oral pharyngeal cavity, respectively. They are tongue coating and tonsilloliths.

Tongue coating is a bacterial plaque, whitish or yellowish in color, which forms on the posterior part of the tongue. It is composed of epithelial cells peeled off the oral mucosa, salivary proteins and food protein remains, which will serve as food to existing bacteria (anaerobic proteolytic bacteria). As the bacterial metabolism ends, bad smelling compounds are produced, called Volatile Sulfur Compounds (VSCs), which are gases derived from sulfur: Sulfur hydride (SH₂), Methyl mercaptane (CH₃SH), and Dimethyl sulfide [(CH₃)2S].

Such compounds, once they reach a certain concentration, give breath a characteristic smell of sulfur or rotten egg, which is nothing but bad breath itself.

Tongue coating is basically formed when one faces a reduction of salivary flow or an epithelial desquamation above physiological limits, or even both situations. There are several causes for increasing cell desquamation, among them desiccation caused by oral respiration or snoring, frequent ingestion of alcoholic beverages or even the use of mouthwashes containing alcohol, the use of orthodontic appliances and the habit of biting lips and cheeks, among others.

A reduction in saliva mainly occurs due to stress and use of medications that diminish the saliva production as a side effect, as well as the low ingestion of liquids, besides other less frequent causes.

Tonsillolith is a “little paste” that is formed within invaginations that exist in the tonsils (tonsil crypts). Its composition is similar to tongue coating (see explanation above), and occurs due to the same mechanism, i.e., epithelial desquamation and reduction of salivary flow. It can be expelled during talking, coughing or sneezing. It is a viscous mass and its name is derived from Latin for “caseum”, which means cheese, since it looks like a little “cheese ball” with an extremely unpleasant odor.

Products used for mouth rinsing and gargling usually contain alcohol in their formulation, which dehydrates and desiccates oral mucosa, increasing cell desquamation, consequently increasing the formation of tongue coating and tonsilloliths, since proteolytic bacteria decompose these peeled off cells, originating Volatile Sulfur Compounds (VSCs) in such process.

In view of this ascertainment, the mouthwash of this patent was developed having a non-alcohol content as a feature.

Another factor is that, among bacteria present in tongue coating and tonsilloliths, there are bacteria that may cause systemic diseases, such as gastritis and pneumonia, as well as may cause dental cavities and periodontal disease, the latter of which also causes a number of systemic diseases. Therefore, removal and control of tongue coating and tonsilloliths are important tools for preventing systemic diseases, dental cavities and periodontal disease.

Another factor is that nowadays the existing procedures for avoiding the formation of tonsilloliths consist of a surgery to extract tonsils and, more recently, of laser surgery, aiming for obliterating or removing tonsil crypts, thus leaving a remainder of tonsils, while having the inconvenience of a very painful postoperative period, at times making it necessary to perform more than one surgical intervention to solve the problem.

### SUMMARY

In light of this ascertainment, regarding mouthwashes with alcohol content, one aspect described herein includes a mouthwash comprising an active ingredient group in respect to tonsilloliths, whose main effect is to oxygenate the little cavities existing in tonsils (crypts), enabling through gargling their cleansing, by releasing the tonsilloliths and, through periodic use, contributing to avoid its new formations, since the existing bacteria are anaerobic, requiring a minimally oxygenated environment to proliferate.

Regarding tongue coating, the active ingredient's effect includes oxygenating the microscopic spaces existing between lingual papillae, facilitating the removal of tongue coating by using a tongue cleaner and/or a toothbrush. Furthermore, through its residual effect, the active ingredient may also contribute to a decrease in new formations through periodic oxygenation of these micro-spaces located among lingual papillae.

The pH adjustment group is intended to allow the determination of the product's pH, and also works as a Hydrogen peroxide stabilizer.

The flavorizer group includes an effect to give the product a pleasant flavor and hence, provide a pleasant odor to the mouthwash user's breath.

The preservative group comprises substances added to oral hygiene and personal hygiene products, cosmetics and perfumes, having a purpose including preserving such products from damage and/or deterioration caused by microorganisms during manufacture and storage, as well as protecting consumers from inadvertent contamination during use of the product.

The bactericide group allows a reduction in the accumulation of bacterial plaque and prevents gingivitis.

The colorant group allows coloring the product.

The additives group comprises similar additives used in oral hygiene products to prevent dental cavities.

The group of solubilizers facilitates mixing flavorizers with solubilizer media.

The antifoamer group allows for a decrease in the formation of foam when using the product and for an increase in solubilizer concentration.

The edulcorant group allows sweetening the product, giving it a pleasant flavor to the palate.

The solubilizer media group acts as the miscibilizer for all of the above ingredients.

In an aspect, an alcohol-free compound includes a mouthwash developed to decrease or avoid the formation of tonsilloliths, to facilitate the removal and decrease new formations of tongue coating, as well as to reduce the concentration and formation of volatile sulfur compounds. In one aspect, the compound includes Hydrogen peroxide, Citric acid, flavorizer, Sodium benzoate, Cetylpyridinium chloride, colorant, Sodium fluoride, Methyl paraben, Polisorbate 20, Dimethicone, Sodium saccharine, Sorbitol and Demineralized water.

The compound may be used in the form of mouth washing and gargling, thereby releasing its active principles.

### DETAILED DESCRIPTION

In one aspect, mouthwash formulations include at least eleven different types of ingredients, including, but not limited to, ingredient types with strong oxidant effect due to oxygen liberation, ingredient types with a flavorizing effect, ingredient types with a bactericide effect and ingredient types with an additive effect.

In order to obtain a desired appearance and flavor, aspects may further include edulcorant, solubilizer, and colorant ingredients.

In order to facilitate the product's use and stability, aspects may further include pH adjusting ingredients, preservatives and antifoamer ingredients.

These ingredients may be included in an aqueous-based media, so that active agents may be conducted to the oral cavity.

The group of ingredients having a strong oxidant effect, due to the freeing of oxygen, includes, but is not limited to, Hydrogen peroxide, Sodium monohydrate perborate and Chlorine dioxide (Sodium chlorite/Sodium chlorate). In one preferred aspect, the ingredient having a strong oxidant effect comprises Hydrogen peroxide.

The ingredient group having a strong oxidant effect may be present in the formulation in an amount from about 0.05% to about 3% of the volume, or preferably from about 0.1% to about 2% of the volume, or preferably from about 0.3% to about 1% of the volume, or preferably in about 0.6% of the volume of the whole formulation.

The group of ingredients having a flavorizing effect includes, but is not limited to, Zinc citrate, Zinc chloride, Tutti frutti, Menthol, Methyl salicylate, eucalyptus oil, spearmint oil, and peppermint oil. In one preferred aspect, a variety of ingredients with a flavorizing effect may be used to allow for a broad range of flavor options.

The ingredient group having a flavorizing effect can be found in the formulation in about 0.01% to about 1% of the volume, or preferably in about 0.05% to about 0.5% of the volume, or preferably in about 0.075% to about 0.3% of the volume, or preferably in about 0.15% of the volume of the whole formulation.

The group of ingredients having a bactericide effect includes, but is not limited to, Cetylpyridinium chloride, Delmopinol, Benzalkonium chloride, Sodium bicarbonate, Chlorhexidine gluconate, Chlorhexidine digluconate, Chlorine dioxide (Sodium chlorite/Sodium chlorate), Triclosan, Polyhexamethylene biguanide chlorhydrate, Sanguinaria Canadensis, Propolis, Aloe vera, Sage (Salvia officinalis), Lemon (Citrus limon), Pine (Pinus sylvestris), Echinacea (Echinacea purpurea and angustifolia), Rathany (Krameria trianda) and Cheeseweed mallow (Malva parviflora L.). In one preferred aspect, the bactericide ingredient comprises Cetylpyridinium chloride.

The ingredient group having a bactericide effect can be found in the formulation in about 0.01% to about 0.5% of the volume, or preferably from about 0.02% to about 0.25% of the volume, or preferably in about 0.03% to about 0.1% of the volume, or preferably in about 0.05% of the volume of the whole formulation.

The group of ingredients having an additive effect include, but are not limited to, Sodium fluoride.

The ingredient group having an additive effect can be found in the formulation in about 0.01% to about 0.5% of the volume, or preferably in about 0.02% to about 0.25% of the volume, or preferably in about 0.03% to about 0.1% of the volume, or preferably in about 0.05% of the volume of the whole formulation.

The group of ingredients used to obtain ideal appearance and flavor includes, but is not limited to, ingredients with an edulcorant, solubilizer, or a colorant effect.

The ingredient group having an edulcorant effect includes, but is not limited to, Sodium saccharine, Sorbitol, Xylitol, Aspartame, Sodium cyclamate, and Stevia. In one preferred aspect, the ingredients having an edulcorant effect comprise Sodium saccharine or Sorbitol.

The group of ingredients having an edulcorant effect, more specifically Sodium saccharine, may be present in the formulation in a quantity of about 0.01% to about 1% of the volume, or preferably in about 0.02% to about 0.5% of the volume, or preferably in about 0.04% to about 0.1% of the volume, or preferably in about 0.05% of the volume of the whole formulation.

The group of ingredients having an edulcorant effect, more specifically Sorbitol, may be present in the formulation in a quantity of about 0.5% to about 20% of the volume, or preferably in about 1% to about 15% of the volume, or preferably in about 3% to about 10% of the volume, or preferably in about 4% of the volume of the whole formulation.

The ingredient group having a solubilizer effect includes, but is not limited to, Polisorbate 20 (Tween 20), Propylene glycol, Polyoxyl 40 and a solubilizer that mixes Propylene glycol, Polyethylene glycol and water. In one preferred aspect, the solubilizer ingredient comprises Polisorbate 20.

The group of ingredients having a solubilizer effect may be present in the formulation in a quantity of about 0.05% to about 2% of the volume, or preferably in about 0.1% to about 1.5% of the volume, or preferably in about 0.25% to about 1% of the volume, or preferably in about 0.5% of the volume of the whole formulation.

The group of ingredients having a colorant effect includes, but is not limited to, all colorants, for food or oral hygiene products. In one preferred aspect, different ingredients with a colorant effect may be used to provide the product with a range of color options.

The group of ingredients having a colorant effect may be pre-sent in the formulation in a quantity of about 0.0001% to about 1% of the volume, or preferably in about 0.0005% to about 0.1% of the volume, or preferably in about 0.001% to about 0.01% of the volume, or preferably in about 0.002% of the volume of the whole formulation.

The group of ingredients facilitating the use and the stability of the product includes, but is not limited to, pH adjusters, preservatives and antifoamer ingredients.

The group of pH adjuster includes, but is not limited to, boric acid and citric acid. In one preferred aspect, the pH adjuster comprises citric acid.

The group of ingredients having a pH adjusting effect may be present in the formulation in a quantity of about 0.01% to about 1% of the volume, or preferably in about 0.05% to about 0.5% of the volume, or preferably in about 0.1% to about 0.25% of the volume, or preferably in about 0.15% of the volume of the whole formulation.

The group of ingredient having a preservative effect includes, but is not limited to, Sodium benzoate, Nipagin or Methyl paraben, Benzoic acid, Formaldehyde, Thymol and Nipazol or Isopropyl paraben. In one preferred aspect, the preservatives comprise Sodium benzoate or Methyl paraben.

The group of ingredients having a preservative effect, more specifically Methyl paraben, may be present in the formulation in a quantity of about 0.01% to about 0.5% of the volume, or preferably in about 0.02% to about 0.25% of the volume, or preferably in about 0.03% to about 0.1% of the volume, or preferably in about 0.05% of the volume of the whole formulation.

The group of ingredients having a preservative effect, more specifically Sodium benzoate, may be present in the formulation in a quantity of about 0.01% to about 1.0% of the volume, or preferably in about 0.05% to about 0.5% of the volume, or preferably in about 0.1% to about 0.25% of the volume, or preferably in about 0.15% of the volume of the whole formulation.

The group of antifoamer ingredients includes, but is not limited to, Dimethicone.

The group of antifoamer ingredients may be present in a quantity of about 0.005% to about 0.5% of the volume, or preferably in about 0.075% to about 0.25% of the volume, or preferably in about 0.01% to about 0.1% of the volume, or preferably in about 0.03% of the volume of the whole formulation.

The group of ingredients enabling active agents to be conducted to the oral cavity in an aqueous-based solubilizer media includes, but is not limited to, demineralized water, distilled water, deionized water, and mineral water. In one preferred aspect, the aqueous-based solubilizer media comprises demineralized water;

The invention shall be more clearly understood in the following example, which is intended merely to exemplify and not to limit the invention.

### EXAMPLE

| Raw Material | % | Function |
|---|---|---|
| Citric acid | 0.15 | pH adjustment |
| Hydrogen peroxide | 0.60 | Active |
| Flavorizer | 0.15 | Flavor |
| Sodium benzoate | 0.15 | Preservative |
| Cetylpyridinium chloride | 0.05 | Bactericide |
| Colorant | 0.002 | Colorant |
| Sodium fluoride | 0.05 | Additive |
| Methyl paraben | 0.05 | Preservative |
| Polisorbate 20 | 0.5 | Solubilizer |
| Sodium saccharine | 0.05 | Edulcorant |
| Dimethicone | 0.03 | Antifoamer |
| Sorbitol | 4 | Edulcorant |
| Demineralized water | q.s.p | Solubilizer Media |

Optionally, instead of the mouthwash comprising one ingredient of each group, as in the above example, it may comprise more than one ingredient of each group. Therefore, one of the possibilities would be that the mouthwash's composition might have the group of active ingredients formed, for instance, by association with Hydrogen peroxide and Sodium perborate, and/or the group of antimicrobials formed by an association of Cetylpyridinium chloride and Chlorhexidine and/or others. In other words, the mouthwash might comprise one ingredient of each group, as described above, or more than one ingredient of one of the groups, or more than one ingredient of more than one of the groups, or more than one ingredient of all groups, used in association and in appropriate quantities for the desired preventive or treatment effect.

1.-54. (canceled)

55. A mouthwash for the prevention and treatment of halitosis, comprising:
at least one ingredient from each of the following ingredient groups: an active ingredient; a pH adjuster; a flavorizer; a preservative; a bactericide; a colorant; an additive; a solubilizer; an antifoamer; an edulcorant; a solubilizer media;
wherein:
the active ingredient has an oxidant effect due to the freeing of oxygen and comprises at least one of Hydrogen peroxide, or Sodium monohydrate perborate or Chlorine dioxide (Sodium chlorite/Sodium chlorate);
the pH adjuster comprises at least one of citric acid or boric acid;
the flavorizer includes a flavorizer for oral hygiene products comprising at least one of Zinc citrate, or Zinc chloride, or Tutti frutti, or Menthol, or Methyl salicylate, or eucalyptus oil, or spearmint oil, or peppermint oil;
the preservative comprises at least one of Sodium benzoate, or Nipagin, or Methyl paraben, or Benzoic acid, or Formaldehyde, or Thymol, or Nipazol, or Isopropyl paraben;
the bactericide comprises at least one of Cetylpyridinium chloride, or Benzalkonium chloride, or Delmopinol, or Sodium bicarbonate, or Chlorhexidine gluconate, or Chlorhexidine digluconate, or Chlorine dioxide (Sodium chlorite/Sodium chlorate), or Triclosan, or Polyhexamethylene biguanide chlorhydrate, or Sanguinaria Canadensis, or Propolis, or Aloe vera, or Sage (Salvia officinalis), or Lemon (Citrus limon), or Pine (Pinus sylvestris), or Echinacea (Echinacea purpurea and angustifolia), or Rathany (Krameria trianda), or Cheeseweed mallow (Malva parviflora L.);
the colorant comprises a colorant for food or oral hygiene products;
the solubilizer comprises at least one of Polisorbate 20, or Propylene glycol, or Polyoxyl 40 or a solubilizer mixing Propylene glycol and water or Polyethylene glycol and water;
the antifoamer comprising Dimethicone;
the edulcorant comprising at least one of Sodium saccharine, or Sorbitol, or Xylitol, or Aspartame, or Sodium cyclamate or Stevia; and
the solubilizer media comprising at least one of demineralized water, or distilled water, or deionized water or mineral water.

56. The mouthwash of claim 55, wherein the active ingredient comprises Hydrogen peroxide, and is present in a quantity of about 0.05% to about 3% by volume.

57. The mouthwash of claim 55, wherein the active ingredient is present in a quantity of about 0.1% to about 2% by volume.

58. The mouthwash of claim 55, wherein the active ingredient is present in a quantity of about 0.3% to about 1% by volume.

59. The mouthwash of claim 55, wherein the active ingredient is present in a quantity of about 0.6% by volume.

60. The mouthwash of claim 55, wherein the pH adjuster comprises Citric acid, and is present in a quantity of about 0.01% to about 1% by volume.

61. The mouthwash of claim 55, wherein the pH adjuster is present in a quantity of about 0.05% to about 0.5% by volume.

62. The mouthwash of claim 55, wherein the pH adjuster is present in a quantity of about 0.1% to about 0.25% by volume.

63. The mouthwash of claim 55, wherein the pH adjuster is present in a quantity of about 0.15% by volume.

64. The mouthwash of claim 55, wherein the flavorizer is present in a quantity of about 0.01% to about 1% by volume.

65. The mouthwash of claim 55, wherein the flavorizer is present in a quantity of about 0.05% to about 0.5% by volume.

66. The mouthwash of claim 55, wherein the flavorizer is present in a quantity of about 0.075% to about 0.3% by volume.

67. The mouthwash of claim 55, wherein the flavorizer is present in a quantity of about 0.15% by volume.

68. The mouthwash of claim 55, wherein the preservative comprises Methyl paraben, and is present in a quantity of about 0.01% to about 0.5% by volume.

69. The mouthwash of claim 55, wherein the preservative is present in a quantity of about 0.02% to about 0.25% by volume.

70. The mouthwash of claim 55, wherein the preservative is present in a quantity of about 0.03% to about 0.1% by volume.

71. The mouthwash of claim 55, wherein the preservative is present in a quantity of about 0.05% by volume.

72. The mouthwash of claim 55, wherein the preservative comprises Sodium benzoate, and is present in a quantity of about 0.01% to about 1% by volume.

73. The mouthwash of claim 55, wherein the preservative is present in a quantity of about 0.05% to about 0.5% by volume.

74. The mouthwash of claim 55, wherein the preservative is present in a quantity of about 0.1% to about 0.25% by volume.

75. The mouthwash of claim 55, wherein the preservative is present in a quantity of about 0.15% by volume.

76. The mouthwash of claim 55, wherein the bactericide comprises Cetylpyridinium chloride.

77. The mouthwash of claim 55, wherein the bactericide is present in a quantity of about 0.01% to about 0.5% by volume.

78. The mouthwash of claim 55, wherein the bactericide is present in a quantity of about 0.02% to about 0.25% by volume.

79. The mouthwash of claim 55, wherein the bactericide is present in a quantity of about 0.03% to about 0.1% by volume.

80. The mouthwash of claim 55, wherein the bactericide is present in a quantity of about 0.05% by volume.

81. The mouthwash of claim 55, wherein the colorant is present in a quantity of about 0.0001% to about 1% by volume.

82. The mouthwash of claim 55, wherein the colorant is present in a quantity of about 0.0005% to about 0.1% by volume.

83. The mouthwash of claim 55, wherein the colorant is present in a quantity of about 0.001% to about 0.01% by volume.

84. The mouthwash of claim 55, wherein the colorant is present in a quantity of about 0.002% by volume.

85. The mouthwash of claim 55, wherein the additive comprises Sodium fluoride.

86. The mouthwash of claim 55, wherein the additive is present in a quantity of about 0.01% to about 0.5% by volume.

87. The mouthwash of claim 55, wherein the additive is present in a quantity of about 0.02% to about 0.25% by volume.

88. The mouthwash of claim 55, wherein the additive is present in a quantity of about 0.03% to about 0.1% by volume.

89. The mouthwash of claim 55, wherein the additive is present in a quantity of about 0.05% by volume.

90. The mouthwash of claim 55, wherein the solubilizer comprises Polisorbate 20, and is present in a quantity of about 0.05% to about 2% by volume.

91. The mouthwash of claim 55, wherein the solubilizer is present in a quantity of about 0.1% to about 1.5% by volume.

92. The mouthwash of claim 55, wherein the solubilizer is present in a quantity of about 0.25% to about 1% by volume.

93. The mouthwash of claim 55, wherein the solubilizer is present in a quantity of about 0.5% by volume.

94. The mouthwash of claim 55, wherein the antifoamer comprises Dimethicone, and is present in a quantity of about 0.005% to about 0.5% by volume.

95. The mouthwash of claim 55, wherein the antifoamer is present in a quantity of about 0.075% to about 0.25% by volume.

96. The mouthwash of claim 55, wherein the antifoamer is present in a quantity of about 0.01% to about 0.1% by volume.

97. The mouthwash of claim 55, wherein the antifoamer is present in a quantity of about 0.03% by volume.

98. The mouthwash of claim 55, wherein the edulcorant comprises Sodium saccharine, and is present in a quantity of about 0.01% to about 1% by volume.

99. The mouthwash of claim 55, wherein the edulcorant is present in a quantity of about 0.02% to about 0.5% by volume.

100. The mouthwash of claim 55, wherein the edulcorant is present in a quantity of about 0.04% to about 0.1% by volume.

101. The mouthwash of claim 55, wherein the edulcorant is present in a quantity of about 0.05% by volume.

102. The mouthwash of claim 55, wherein the edulcorant comprises Sorbitol, and is present in a quantity of about 0.5% to about 20% by volume.

103. The mouthwash of claim 55, wherein the edulcorant is present in a quantity of about 1% to about 15% by volume.

104. The mouthwash of claim 55, wherein the edulcorant is present in a quantity of about 2% to about 10% by volume.

105. The mouthwash of claim 55, wherein the edulcorant is present in a quantity of about 4% by volume.

106. The mouthwash of claim 55, further comprising a predetermined group of at least one edulcorant, at least one solubilizer, and at least one colorant corresponding to a desired appearance and flavor.

107. The mouthwash of claim 55, further comprising a predetermined group of at least one pH adjuster, at least one preservative, and at least one antifoamer corresponding to a desired use and stability of the mouthwash.

108. The mouthwash of claim 55, wherein the solubilizer media comprises demineralized water having an aqueous-base operable to conduct the active ingredient to an oral cavity.
